(19) United States
(12) Patent Application Publication
(10) Pub. No.: US 2026/0279497 A1
(43) Pub. Date: Sep. 17, 2026

(54) MACHINE LEARNING-BASED METHOD FOR GENOTYPE-ENVIRONMENT INTERACTION AND APPLICATION OF METHOD

(71) Applicants: Institute of Crop Sciences, Chinese Academy of Agricultural Sciences, Beijing (CN); Sanya National Institute of Southern Breeding, Chinese Academy of Agricultural Sciences, Sanya (CN)

(72) Inventors: Huihui LI, Beijing (CN); Tingxi YU, Beijing (CN); Kunhui HE, Beijing (CN)

(21) Appl. No.: 19/676,597

(22) Filed: May 13, 2026

Related U.S. Application Data
(63) Continuation of application No. PCT/CN2025/091404, filed on Apr. 27, 2025.

(30) Foreign Application Priority Data
Mar. 5, 2024 (CN) 202410245774.4

(51) Int. Cl. G16B 40/00; G06F 30/27; G16B 20/00

(52) U.S. Cl. CPC G16B 40/00; G06F 30/27; G16B 20/00

(57) ABSTRACT

The present invention relates to the field of bioinformatics, and specifically discloses a machine learning-based method for genotype-environment interaction and application thereof, including the following steps: step 1: acquiring environmental data for each growth stage during a crop growth period; step 2: calculating an environmental index for a target growth stage; step 3: calculating a mean environmental index and a comparative mean environmental index, and determining a growth stage environmental index that most affects the mean environmental index, namely, an environmental index with the highest correlation; step 4: calculating a phenotypic plasticity value of a target gene; step 5: calculating an environmental impact parameter of a potential functional gene; and step 6: determining whether the potential functional gene is an environment-influenced important potential functional gene.

### CROSS-REFERENCE TO RELATED APPLICATIONS

The present application is a continuation of International Application No. PCT/CN2025/091404, filed on Apr. 27, 2025, which claims priority to Chinese Patent Application No. 202410245774.4, filed on Mar. 5, 2024, the entire disclosure of which is incorporated herein by reference.

### FIELD OF TECHNOLOGY

The present invention relates to the field of bioinformatics technology, and in particular to a machine learning-based method for genotype-environment interaction and application of the method.

### BACKGROUND

In the field of biology and breeding, particularly crop breeding, phenotypes mean observable characteristics of an organism, such as a shape, a structure, a size, and a color, as determined by a genotype and an environment. A phenome means a complete set of traits of a given organism, which is not limited to agronomic traits but also encompasses a physiological state exhibited by a plant.

The Chinese invention with an authorized patent publication number CN110459265B discloses a method for improving accuracy of genomic prediction (GP). The method includes: (1) performing phenotyping and genotyping on a target crop population, followed by conducting genome-wide association studies (GWAS) on the entire population to identify four single nucleotide polymorphisms (SNPs) with the largest effect size; and (2) using the four SNPs with the largest effect size as fixed effects, and incorporating a genotype-environment interaction (G×E) component into a genomic prediction (GP) model, to improve prediction accuracy to the greatest extent.

Phenotypic variation is determined by genetic factors, environmental factors, and interactions. To breed crops with a high yield and strong adaptability to emerging and variable climates, it is essential to dissect effects of the environmental factors. Although significant progress has been made in improving prediction accuracy, existing genomic and genotype-environment (G×E) prediction models lack interpretability. If relative contributions of the genetic factors and the environmental factors cannot be accurately quantified and underlying factors cannot be identified, multiple long-standing biological questions remain unresolved. Therefore, it is necessary to establish an integrated framework incorporating environmental dimensions for analysis and prediction of complex traits.

### SUMMARY

The present invention is intended to provide a machine learning-based method for genotype-environment interaction and application thereof, to solve the technical problems mentioned in the background.

The objective of the present invention can be achieved in the following technical solutions:

The machine learning-based method for genotype-environment interaction, including the following steps:
Step 1: acquiring environmental data for each growth stage during a crop growth period;
Step 2: calculating an environmental index for a target growth stage based on the environmental data;
Step 3: calculating environmental indices for all growth stages during the crop growth period, calculating a mean environmental index and a comparative mean environmental index, and determining a growth stage environmental index that most affects the mean environmental index, namely, an environmental index with the highest correlation;
Step 4: calculating a phenotypic plasticity value of a target gene based on the environmental index with the highest correlation and a phenotype of the target gene;
Step 5: calculating an environmental impact parameter of a potential functional gene based on the phenotypic plasticity value; and
Step 6: determining, based on the environmental impact parameter of the potential functional gene, whether the potential functional gene is an environment-influenced important potential functional gene; and
if the environmental impact parameter of the potential functional gene is less than a threshold value of the environmental impact parameter of the potential functional gene, it is determined that the potential functional gene is not the environment-influenced important potential functional gene; or
if the environmental impact parameter of the potential functional gene is greater than or equal to the threshold value of the environmental impact parameter of the potential functional gene, it is determined that the potential functional gene is the environment-influenced important potential functional gene.

In a further solution of the present invention, the environmental data includes: an effective accumulated temperature, photosynthetically active radiation, effective moisture, and a soil pH.

In a further solution of the present invention, a specific calculation method for the environmental index is as follows:
labeling an effective accumulated temperature as Wₙ, labeling photosynthetically active radiation as Gₙ, labeling effective moisture as Sn, labeling a soil pH as Tₙ, and performing data processing, where n represents a different growth stage, taking values 1, 2, 3, . . . , R, and R is a positive integer;
calculating the environmental index Zₙ by using a formula

$${Z}_{n} = {a}_{1} \times {( \frac{{W}_{n} \times {G}_{n} \times {S}_{n} \times {T}_{n}}{{a}_{2}} )}^{2} + ( \frac{{W}_{n} \times {G}_{n} \times {S}_{n} \times {T}_{n}}{{a}_{3}} ) + {a}_{4} ,$$

where a₁, a₂, a₃, a₄ are preset ratio factors, and a₁, a₂, a₃ are all not equal to 0.

In a further solution of the present invention, a specific calculation method for the mean environmental index is as follows:
A1: presetting the environmental index with the highest correlation as Zᵢ, where i=1, 2, 3, . . . , R, and R is a positive integer;
A2: calculating the mean environmental index based on environmental indices for all growth stages during the growth period; and
calculating the mean environmental index Z̅ by using a formula

$$\overline{Z} = \frac{{\sum }_{1}^{n} {Z}_{n}}{n} ,$$

where n represents a different growth stage.

In a further solution of the present invention, a specific calculation method for the comparative mean environmental index is as follows:
calculating the comparative mean environmental index Z̅ᵢ̅ based on the mean environmental index Z̅ and by using a formula

$$\overline{{Z}_{i}} = \frac{{\sum }_{1}^{n} {Z}_{n} - {Z}_{i}}{n - 1} ,$$

where n represents a different growth stage.

In a further solution of the present invention, a method for determining the environmental index with the highest correlation is as follows:
calculating a difference between the mean environmental index Z̅ and the comparative mean environmental index Z̅ᵢ̅ to obtain an index difference, and analyzing and comparing the index difference to determine the environmental index with the highest correlation, where the environmental index with the highest correlation is a set with a maximum index difference.

In a further solution of the present invention, obtaining the phenotypic plasticity value of the target gene using the least squares method based on the environmental index with the highest correlation and the phenotype of the target gene specifically includes:
B1: changing the environmental index Zₑ with the highest correlation to obtain different phenotypes of the target gene, and labeling the phenotypes of the target gene as Xₑ, where e represents a different environmental index, taking values 1, 2, 3, . . . , R, and R is a positive integer; and
B2: based on multiple sets of data points (Z₁, X₁), (Z₂, X₂), . . . , (Zₑ, Xₑ), finding a line, so that a sum of vertical distances from all data points to this line is minimized, and then this line is the phenotypic plasticity value of the target gene.

In a further solution of the present invention, a specific calculation method for the environmental impact parameter of the potential functional gene is as follows:
C1: obtaining potential functional genes of the target gene, and labeling the potential functional genes as Dⱼ, where j represents a different potential functional gene, taking values 1, 2, 3, . . . , R, and R is a positive integer;
and the potential functional genes include: a gene sequence, a haplotype, SNP (single nucleotide polymorphism);
C2: within a calibration range, changing the environmental index Zₑ with the highest correlation, and recording a ratio H_{D}_{ⱼ} of the number of changes in the potential functional gene and a total amplitude F_{D}_{ⱼ} of changes when the potential functional gene changes;
where the calibration range is a range of changes in the environmental index with the highest correlation, so that the phenotype of the target gene exhibits only a single change; and
the ratio of the number of changes in the potential functional gene is a ratio of the number of changes in the potential functional gene to the number of changes in the environmental index with the highest correlation; and
C3: processing the ratio H_{D}_{ⱼ} of the number of changes in the potential functional gene and the total amplitude F of changes when the potential functional gene changes, and by using a formula Y_{D}_{ⱼ}=b₁×H_{D}_{ⱼ}+b₂×F_{D}_{ⱼ}, calculating the environmental impact parameter Y_{D}_{ⱼ} of the potential functional gene, where b₁ and b₂ are weighting ratio factors, all greater than 0.

In a further solution of the present invention, presetting the threshold value of the environmental impact parameter of the potential functional gene as Yl, and comparing and analyzing the environmental impact parameter Y_{D}_{ⱼ} of the potential functional gene with the threshold value Yl of the environmental impact parameter of the potential functional gene to determine whether the potential functional gene is the environment-influenced important potential functional gene; and
if Y_{D}_{ⱼ} is less than Yl, it indicates that an environment has little effect on the potential functional gene, and it is determined that the potential functional gene is not the environment-influenced important potential functional gene; or
if Y_{D}_{ⱼ} is greater than or equal to Yl, it indicates that an environment has a significant effect on the potential functional gene, and it is determined that the potential functional gene is the environment-influenced important potential functional gene.

### Beneficial Effects of the Present Invention

(1) according to the present invention, environmental information is fully mined by utilizing an artificial intelligence algorithm to analyze phenotypic plasticity of important agronomic traits during key growth stages, elucidate the interaction between genes and the environment, and predict phenotypes of important agronomic traits; and
(2) according to the present invention, genotype-environment interactions are utilized to breed varieties adapted to climate change, match a genotype with an environmental type, identify key factors influencing crop growth and phenotypic variation, develop a cross-environment prediction strategy, optimize variety selection pathways, and assist breeders in making informed production decisions, thereby advancing the plant breeding process.

### BRIEF DESCRIPTION OF THE DRAWINGS

The present invention is further described with reference to accompanying drawings.

FIG. 1 is a schematic diagram of steps of a method according to the present invention;

FIG. 2 is a diagram of the least squares method according to the present invention; and

FIG. 3 is a schematic diagram of steps for determining an environment-influenced important potential functional gene according to the present invention.

### DESCRIPTION OF THE EMBODIMENTS

The following clearly and completely describes technical solutions in embodiments of the present invention with reference to accompanying drawings in the embodiments of the present invention. Apparently, the described embodiments are merely some but not all of the embodiments of the present invention. All other embodiments obtained by a person of ordinary skill in the art based on the embodiments of the present application without creative efforts shall fall within the protection scope of the present application.

### Embodiment 1

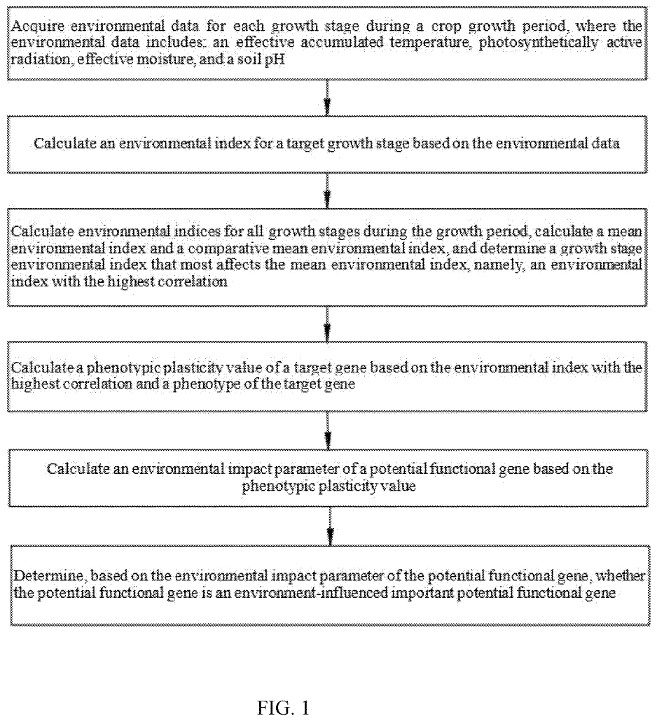

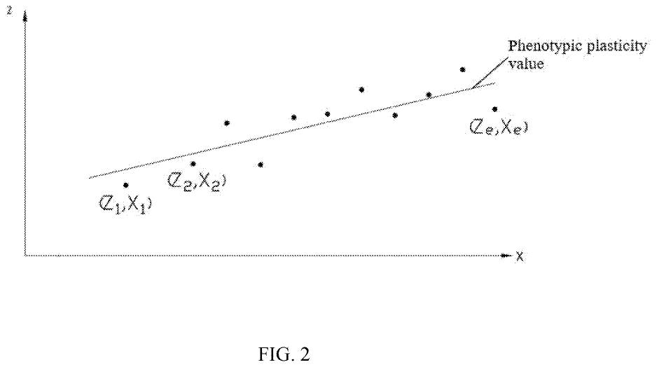

Refer to FIG. 1 and FIG. 2. The present invention provides a machine learning-based method for genotype-environment interaction, including the following steps.

Step 1: acquiring environmental data for each growth stage during a crop growth period.

The environmental data includes: an effective accumulated temperature, photosynthetically active radiation, effective moisture, and a soil pH; and

labeling an effective accumulated temperature as Wₙ, labeling photosynthetically active radiation as Gₙ, labeling effective moisture as Sn, and labeling a soil pH as Tₙ, where n represents a different growth stage, taking values 1, 2, 3, . . . , R, and R is a positive integer.

It should be noted that the crop growth period means a period from sowing to seed maturity, expressed in days required. For some crops such as fiber crops, tuber crops, sugarcane, green manure, and the like, the growth stage means a period from sowing to harvest of a main product.

The growth stage means different growth phases of a crop, divided into several stages according to a sequence of organ development and morphological characteristics during an entire growth process. For example, a growth stage of winter wheat includes an emergence stage, a three-leaf stage, a tillering stage, an overwintering stage, a regreening stage, a jointing stage, a booting stage, a heading stage, a flowering stage, and a maturity stage.

The effective accumulated temperature means a sum of effective temperatures during the growth stage of the crop, namely, a sum of a difference between a daily average temperature and a biological zero point during the growth stage. The effective accumulated temperature reflects a heat requirement for biological growth and development.

The photosynthetically active radiation means solar radiation that can be used by green plants for photosynthesis, with a wavelength range of 380-710 nm. The photosynthetically active radiation is a main energy source for biomass formation and a key factor affecting crop photosynthesis.

The effective moisture means water content in soil that can be absorbed and utilized by crops. Excessive or insufficient water in the soil affects crop growth and yield.

The soil pH means a degree of acidity or alkalinity of the soil. The Soil pH is one of important factors affecting soil fertility, which affects not only the availability of soil nutrients but also the activity of microorganisms in the soil, thereby affecting crop growth and yield.

Step 2: Calculating an environmental index for a target growth stage based on the environmental data, with a specific calculation method as follows:

processing the effective accumulated temperature, the photosynthetically active radiation, the effective moisture, and the soil pH, and calculating the environmental index Zₙ by using a formula

$${Z}_{n} = {a}_{1} \times {( \frac{{W}_{n} \times {G}_{n} \times {S}_{n} \times {T}_{n}}{{a}_{2}} )}^{2} + ( \frac{{W}_{n} \times {G}_{n} \times {S}_{n} \times {T}_{n}}{{a}_{3}} ) + {a}_{4} ,$$

where a₁, a₂, a₃, a₄ are preset ratio factors, and a₁, a₂, a₃ are all not equal to 0.

Step 3: Calculating environmental indices for all growth stages during the crop growth period, calculating a mean environmental index and a comparative mean environmental index, and determining a growth stage environmental index that most affects the mean environmental index, namely, an environmental index with the highest correlation, with a specific method as follows:
A1: presetting the environmental index with the highest correlation as Zᵢ, where i=1, 2, 3, . . . , R, and R is a positive integer;
A2: calculating the mean environmental index based on environmental indices for all growth stages during the growth period; and
calculating the mean environmental index Z̅ by using a formula

$$\overline{Z} = \frac{{\sum }_{1}^{n} {Z}_{n}}{n} ,$$

where n represents a different growth stage;
A3: calculating the comparative mean environmental index with the environmental index with the highest removed;
calculating the comparative mean environmental index Z̅ᵢ̅ based on the mean environmental index Z̅ by using a formula

$$\overline{{Z}_{i}} = \frac{{\sum }_{1}^{n} {Z}_{n} - {Z}_{i}}{n - 1} ,$$

where n represents a different growth stage; and
A4: calculating a difference between the mean environmental index Z̅ and the comparative mean environmental index Z̅ᵢ̅ to obtain an index difference, and analyzing and comparing the index difference to determine the environmental index with the highest correlation, where the environmental index with the highest correlation is a set with a maximum index difference;
Step 4: obtaining a phenotypic plasticity value of the target gene using the least squares method based on the environmental index with the highest correlation and a phenotype of a target gene, where the method specifically includes:
B1: changing the environmental index Zₑ with the highest correlation to obtain different phenotypes of the target gene, and labeling the phenotypes of the target gene as Xₑ, where e represents different environmental indices, taking values 1, 2, 3, . . . , R, and R is a positive integer; and
B2: based on multiple sets of data points (Z₁, X₁), (Z₂, X₂), . . . , (Zₑ, Xₑ), finding a line, so that a sum of vertical distances from all data points to this line is minimized, and then this line is the phenotypic plasticity value of the target gene.

### Embodiment 2

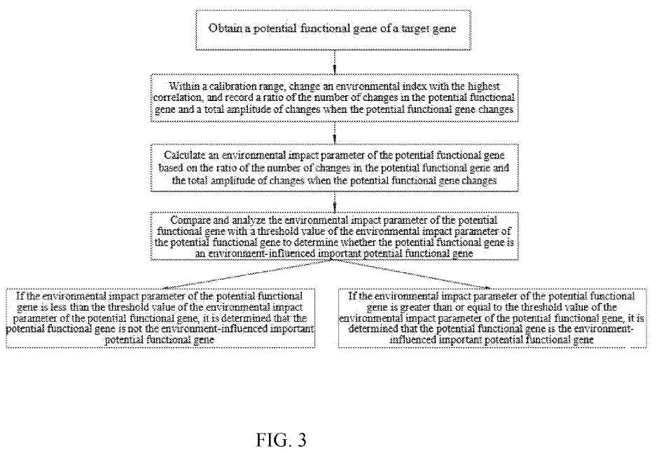

Based on Embodiment 1, as shown in FIG. 3, the present invention provides a machine learning-based method for genotype-environment interaction, further including: calculating an environmental impact parameter of a potential functional gene according to a phenotypic plasticity value, and determining whether the potential functional gene is an environment-influenced important potential functional gene,
determining whether the potential functional gene is the environment-influenced important potential functional gene is intended to identify a key factor affecting crop growth and phenotypic variation, thereby formulating a cross-environment prediction strategy, optimizing a variety selection path, and assisting a breeder in making a production decision, thus promoting a plant breeding process;
C1: obtaining potential functional genes of the target gene, and labeling the potential functional genes as Dⱼ, where j represents different potential functional genes, taking values 1, 2, 3, . . . , R, and R is a positive integer;
and the potential functional genes include: a gene sequence, a haplotype, SNP (single nucleotide polymorphism);
C2: within a calibration range, changing the environmental index Zₑ with the highest correlation, and recording a ratio H_{D}_{ⱼ} of the number of changes in the potential functional gene and a total amplitude F_{D}_{ⱼ} of changes when the potential functional gene changes;
where the calibration range is a range of changes in the environmental index with the highest correlation, so that the phenotype of the target gene exhibits only a single change; and
the ratio of the number of changes in the potential functional gene is a ratio of the number of changes in the potential functional gene to the number of changes in the environmental index with the highest correlation;
C3: processing the ratio of the number of changes in the potential functional gene H_{D}_{ⱼ} and the total amplitude F of changes when the potential functional gene changes, and by using a formula Y_{D}_{ⱼ}=b₁×H_{D}_{ⱼ}+b₂× F_{D}_{ⱼ}, calculating the environmental impact parameter Y_{D}_{ⱼ}, of the potential functional gene, where b₁ and b₂ are weighting ratio factors, all greater than 0; and
C4: presetting a threshold value of the environmental impact parameter of the potential functional gene as Yl, and comparing and analyzing the environmental impact parameter Y_{D}_{ⱼ} of the potential functional gene with the threshold value Yl of the environmental impact parameter of the potential functional gene to determine whether the potential functional gene is the environment-influenced important potential functional gene; and
if Y_{D}_{ⱼ} is less than Yl, it indicates that an environment has little effect on the potential functional gene, and it is determined that the potential functional gene is not the environment-influenced important potential functional gene; or
if Y_{D}_{ⱼ} is greater than or equal to Yl, it indicates that an environment has a significant effect on the potential functional gene, and it is determined that the potential functional gene is the environment-influenced important potential functional gene.

### Embodiment 3

Application of the machine learning-based method for genotype-environment interaction to environmental processing

Operating principle of the present invention: Step 1: acquiring environmental data for each growth stage during a crop growth period; Step 2: calculating an environmental index for a target growth stage based on the environmental data; Step 3: calculating environmental indices for all growth stages during the crop growth period, calculating a mean environmental index and a comparative mean environmental index, and determining a growth stage environmental index that most affects the mean environmental index, namely, an environmental index with the highest correlation; Step 4: calculating a phenotypic plasticity value of a target gene based on the environmental index with the highest correlation and a phenotype of the target gene; Step 5: calculating an environmental impact parameter of a potential functional gene based on the phenotypic plasticity value; and Step 6: determining, based on the environmental impact parameter of the potential functional gene, whether the potential functional gene is an environment-influenced important potential functional gene; and if the environmental impact parameter of the potential functional gene is less than a threshold value of the environmental impact parameter of the potential functional gene, it is determined that the potential functional gene is not the environment-influenced important potential functional gene; or if the environmental impact parameter of the potential functional gene is greater than or equal to the threshold value of the environmental impact parameter of the potential functional gene, it is determined that the potential functional gene is the environment-influenced important potential functional gene.

The above provides a detailed description of one embodiment of the present invention, but the content described is only a preferred embodiment and should not be construed as limiting the scope of implementation of the present invention. Any equivalent changes, improvements, or the like made within the scope of the claims of the present invention shall still fall within a scope covered by the present patent.

What is claimed is:

1. A machine learning-based method for genotype-environment interaction, comprising the following steps:
step 1: acquiring environmental data for each growth stage during a crop growth period;
step 2: calculating an environmental index for a target growth stage based on the environmental data;
step 3: calculating environmental indices for all growth stages during the crop growth period, calculating a mean environmental index and a comparative mean environmental index, and determining a growth stage environmental index that most affects the mean environmental index, namely, an environmental index with the highest correlation;
step 4: calculating a phenotypic plasticity value of a target gene based on the environmental index with the highest correlation and a phenotype of the target gene;
step 5: calculating an environmental impact parameter of a potential functional gene based on the phenotypic plasticity value; and
step 6: determining, based on the environmental impact parameter of the potential functional gene, whether the potential functional gene is an environment-influenced important potential functional gene; and
if the environmental impact parameter of the potential functional gene is less than a threshold value of the environmental impact parameter of the potential functional gene, it is determined that the potential functional gene is not the environment-influenced important potential functional gene; or
if the environmental impact parameter of the potential functional gene is greater than or equal to the threshold value of the environmental impact parameter of the potential functional gene, it is determined that the potential functional gene is the environment-influenced important potential functional gene.

2. The machine learning-based method for genotype-environment interaction according to claim 1, wherein the environmental data comprises: an effective accumulated temperature, photosynthetically active radiation, effective moisture, and a soil pH.

3. The machine learning-based method for genotype-environment interaction according to claim 1, wherein a specific calculation method for the environmental index is as follows:
labeling an effective accumulated temperature as Wₙ, labeling photosynthetically active radiation as Gₙ, labeling effective moisture as Sn, labeling a soil pH as Tₙ, and performing data processing, wherein n represents a different growth stage, taking values 1, 2, 3, . . . , R, and R is a positive integer; and
calculating the environmental index Zₙ by using a formula
$${Z}_{n} = {a}_{1} \times {( \frac{{W}_{n} \times {G}_{n} \times {S}_{n} \times {T}_{n}}{{a}_{2}} )}^{2} + ( \frac{{W}_{n} \times {G}_{n} \times {S}_{n} \times {T}_{n}}{{a}_{3}} ) + {a}_{4} ,$$
wherein a₁, a₂, a₃, a₄ are preset ratio factors, and a₁, a₂, a₃ are all not equal to 0.

4. The machine learning-based method for genotype-environment interaction according to claim 3, wherein a specific calculation method for the mean environmental index is as follows:
A1: presetting the environmental index with the highest correlation as Zᵢ, wherein i=1, 2, 3, . . . R, and R is a positive integer;
A2: calculating the mean environmental index based on environmental indices for all growth stages during the growth period; and
calculating the mean environmental index Z̅ by using a formula
$$\overline{Z} = \frac{{\sum }_{1}^{n} {Z}_{n}}{n} ,$$
wherein n represents a different growth stage.

5. The machine learning-based method for genotype-environment interaction according to claim 4, wherein a specific calculation method for the comparative mean environmental index is as follows:
calculating the comparative mean environmental index Z̅ᵢ̅ based on the mean environmental index Z̅ and by using a formula
$$\overline{{Z}_{i}} = \frac{{\sum }_{1}^{n} {Z}_{n} - {Z}_{i}}{n - 1} ,$$
wherein n represents a different growth stage.

6. The machine learning-based method for genotype-environment interaction according to claim 5, wherein a method for determining the environmental index with the highest correlation is as follows:
calculating a difference between the mean environmental index Z̅ and the comparative mean environmental index Z̅ᵢ̅ to obtain an index difference, and analyzing and comparing the index difference to determine the environmental index with the highest correlation, wherein the environmental index with the highest correlation is a set with a maximum index difference.

7. The machine learning-based method for genotype-environment interaction according to claim 6, wherein obtaining the phenotypic plasticity value of the target gene using the least squares method based on the environmental index with the highest correlation and the phenotype of the target gene specifically comprises:
B1: changing the environmental index Zₑ with the highest correlation to obtain different phenotypes of the target gene, and labeling the phenotypes of the target gene as Xₑ, wherein e represents a different environmental index, e is 1, 2, 3, . . . , R, and R is a positive integer; and
B2: based on multiple sets of data points (Z₁, X₁), (Z₂, X₂), . . . , (Zₑ, Xₑ), finding a line, so that a sum of vertical distances from all data points to this line is minimized, and then this line is the phenotypic plasticity value of the target gene.

8. The machine learning-based method for genotype-environment interaction according to claim 1, wherein a specific calculation method for the environmental impact parameter of the potential functional gene is as follows:
C1: obtaining potential functional genes of the target gene, and labeling the potential functional genes as Dⱼ, wherein j represents different potential functional genes, taking values 1, 2, 3, . . . , R, R is a positive integer;
wherein the potential functional genes comprise: a gene sequence, a haplotype, SNP;
C2: within a calibration range, changing the environmental index Zₑ with the highest correlation, and recording a ratio of the number of changes in the potential functional gene H_{D}_{ⱼ} and a total amplitude F_{D}_{ⱼ} of changes when the potential functional gene changes;
wherein the calibration range is a range of changes in the environmental index with the highest correlation, so that the phenotype of the target gene exhibits only a single change; and
the ratio of the number of changes in the potential functional gene is a ratio of the number of changes in the potential functional gene to the number of changes in the environmental index with the highest correlation; and
C3: processing the ratio Hp, of the number of changes in the potential functional gene and the total amplitude F of changes when the potential functional gene changes, and by using a formula Y_{D}_{ⱼ}=b₁×H_{D}_{ⱼ}+b₂×F_{D}_{ⱼ}, calculating the environmental impact parameter Y_{D}_{ⱼ} of the potential functional gene, wherein b₁ and b₂ are weighting ratio factors, all greater than 0.

9. The machine learning-based method for genotype-environment interaction according to claim 1, wherein presetting the threshold value of the environmental impact parameter of the potential functional gene as Yl, and comparing and analyzing the environmental impact parameter Y_{D}_{ⱼ} of the potential functional gene with the threshold value Yl of the environmental impact parameter of the potential functional gene to determine whether the potential functional gene is the environment-influenced important potential functional gene; and
if Y_{D}_{ⱼ} is less than Yl, it indicates that an environment has little effect on the potential functional gene, and it is determined that the potential functional gene is not the environment-influenced important potential functional gene; or
if Y_{D}_{ⱼ} is greater than or equal to Yl, it indicates that an environment has a significant effect on the potential functional gene, and it is determined that the potential functional gene is the environment-influenced important potential functional gene.

10. Application of the machine learning-based method for genotype-environment interaction to environmental processing according to claim 1.
